(19) United States
(12) Patent Application Publication
(10) Pub. No.: US 2024/0108202 A1
(43) Pub. Date: Apr. 4, 2024

(54) ENDOSCOPIC BITE BLOCK

(71) Applicant: University of Kansas, Lawrence, KS (US)

(72) Inventors: Megan Folsom, Overland Park, KS (US); Kaitlyn Witt, Boerne, TX (US); Harshit Gandhi, Parsippany, NJ (US); Titiksha Sharma, Parsippany, NJ (US); Hira Ubaid, Tomball, TX (US)

(21) Appl. No.: 18/478,347

(22) Filed: Sep. 29, 2023

Related U.S. Application Data
(60) Provisional application No. 63/411,996, filed on Sep. 30, 2022.

(51) Int. Cl. A61B 1/00; A61B 1/273; A61M 16/06

(52) U.S. Cl. CPC A61B 1/00154; A61B 1/273; A61M 16/0672; A61M 16/0683

(57) ABSTRACT

A bite block includes a base and a probe opening through the base. Upper and lower biting platforms are connected to the base above and below the probe opening respectively. A nasal cannula holder is connected to an upper portion of the base. A first and second fluid passage each have a proximal end at the base and a distal end extending from the base. The distal ends extend from the base such that, when the bite block is inserted into the mouth of a patient, the distal ends extend to the throat of the patient. A first and second fluid path are each defined between the proximal and distal ends of the first and second fluid passages respectively.

### CROSS-REFERENCE TO RELATED APPLICATIONS

This application claims priority to and the benefit of U.S. Provisional Patent Application No. 63/411,996, filed on Sep. 30, 2022, which is hereby incorporated by reference in its entirety.

### BACKGROUND

### Background and Relevant Art

Gastrointestinal endoscopy is a procedure performed by physicians for examining the upper part of a patient's gastrointestinal tract. This procedure is usually performed to investigate symptoms of upper abdominal pain, nausea, vomiting, difficulty in swallowing, or bleeding in the upper gastrointestinal tract. To perform gastrointestinal endoscopy, a physician inserts endoscopic and/or endotracheal instruments or tools into a patient's mouth. Insertion and/or use of the tools may damage a portion of the patient's mouth or throat. Additionally, or alternatively, the patient may bite down on the instrument or the tool, thereby damaging the instrument or the tool. Bite blocks are often used to protect the patient's mouth and/or the instrument or the tool during an endoscopic or an endotracheal procedure.

Patients undergoing gastrointestinal endoscopy may experience hypoxia, or the oxygen saturation levels in their blood may fall below a certain threshold. The effects of hypoxia can be serious, and in some cases, even dangerous to health. For this reason, a patient may be given oxygen to maintain their oxygen saturation at an acceptable level. Oxygen may be given through a nasal cannula or delivered directly to the airway in a posterior portion of the oral cavity.

It is sometimes difficult to insert an endoscope. In some instances, a patient may be medicated and/or sedated in order to undergo this procedure. Due to this, the patient may relax their muscles, and the patient's tongue will sometimes relax into the back of their mouth, blocking their throat entirely. Additionally, patients undergoing oral endoscopic procedures also have increased salivation creating excessive secretions that can become life-threatening by triggering laryngospasm, or a forced closing of the vocal cords.

### BRIEF SUMMARY

In some embodiments, a bite block for use during an endoscopic operation includes a base and a probe opening through the base. An upper biting platform is connected to an upper portion of the base. A lower biting platform is connected to a lower portion of the base. A nasal cannula holder is configured to retain a nasal cannula, and connected to the upper portion of the base. A first fluid passage has a first proximal end at the base and a first distal end extending from the base such that, when the bite block is in use with a patient, the first distal end is configured to extend to a throat of said patient, the first fluid passage defining a first fluid path extending between the first proximal end and the first distal end. A second fluid passage has a second proximal end at the base and a second distal end extending from the base such that, when the bite block is in use with said patient, the second distal end is configured to extend to the throat of said patient, the second fluid passage defining a second fluid path extending between the second proximal end and the second distal end.

In some embodiments, a method for treating a patient includes inserting a bite block into a mouth of said patient, the bite block including a base, a probe opening, a nasal cannula holder configured to retain a nasal cannula, the nasal cannula holder connected to an upper portion of the base, and a fluid passage. A first flow of oxygen is provided to said patient through the nasal cannula connected to the nasal cannula holder. A second flow of oxygen is provided to said patient through the fluid passage, wherein the second flow of oxygen is distinct from the first flow of oxygen.

In some embodiments, a bite block for use during an endoscopic operation includes a base and a probe opening through the base. An upper biting platform is connected to an upper portion of the base. A lower biting platform is connected to a lower portion of the base. A nasal cannula holder is configured to retain a nasal cannula, and is connected to the upper portion of the base. An oral airway includes a first fluid passage, a second fluid passage, an anterior portion, and a posterior portion configured to rest on a base of a tongue of a patient using the bite block.

In some embodiments, a bite block includes a bite block body with a probe opening extending through the bite block body. An upper biting platform is connected to the bite block body and located above the probe opening. A lower biting platform is connected to the bite block body and located below the probe opening. A first auxiliary opening and a second auxiliary opening may be located on the bite block body and extend through the body. The bite block may also include a nasal cannula holder connected to and located on an upper portion of the bite block body. The bite block may further include a first passage and a second fluid passage that each extend through the bite block body.

In other embodiments, a bite block may include an oral airway body that is connected to a bite block body. The oral airway body may be curved and extend in a posterior direction from the bite block body. The oral airway body may contain a first fluid passage and a second fluid passage such that the first fluid passage and the second fluid passage pass through the oral airway body. The oral airway body may also include an anterior portion and a posterior portion. The posterior portion may further be divided into a first portion and a second portion such that the posterior portion has a split or forked shape. The first fluid passage and the second fluid passage may pass through the first portion and the second portion respectively. The first fluid passage may include a first connector for connecting medical oxygen to and delivering oxygen through the first fluid passage. The second fluid passage may contain a second connector for connecting medical suction to and providing suction through the second fluid passage. The second fluid passage may also connect to and deliver oxygen. The first connector may be angled away from the probe opening.

In other embodiments, the bite block may include a nasal cannula located on an upper portion of a base for delivering nasal oxygen. The nasal cannula may be integrally formed with the base and may connect to an oxygen supply for providing a flow of oxygen to a patient.

In other embodiments, a bite block may include an oral airway body that is detachably connected to a bite block body. The oral airway body may include a clip configured to interface with a slot in the bite block body to achieve a detachable connection. The bite block may also detachably connect through a press fit.

In other embodiments, a bite block includes a body with an inner surface and an outer surface. An endoscopy port extends through the outer surface. A first port through the outer surface is located on a first side of the endoscopy port, the first port having a first connector. A second port through the outer surface is located on the first side of the endoscopy port, the second port having a second connector. The second connector is different from the first connector. A third port through the outer surface is located on a second side of the endoscopy port and has the first connector. A fourth port through the outer surface is located on the second side of the endoscopy port and has the second connector.

In other embodiments, a bite block includes a body having an inner surface and an outer surface. An endoscopy port through the outer surface is connected to an endoscopy tube extending past the inner surface for a first distance. A first port through the outer surface is connected to a first tube extending past the inner surface for a second distance. A second port through the outer surface is connected to a second tube extending past the inner surface for a third distance. The second distance is different than the third distance.

In yet other embodiments, a bite block includes a body having an inner surface and an outer surface. An endoscopy port through the outer surface has a first diameter. A first port through the outer surface is located on a first side of the endoscopy port, the first port having a second diameter. A second port through the outer surface is located on the first side of the endoscopy port, the first port having a third diameter. The first diameter is larger than the second diameter or the third diameter. The second diameter is larger than the third diameter. A third port through the outer surface is located on a second side of the endoscopy port and has the second diameter. A fourth port through the outer surface is located on the second side of the endoscopy port and has the third diameter.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1A is a perspective view of a bite block, according to at least one embodiment of the present disclosure;

FIG. 1B is a side view of the bite block of FIG. 1A, according to at least one embodiment of the present disclosure;

FIG. 1C is a perspective view of a bite block body of the bite block of FIG. 1A, according to at least one embodiment of the present disclosure;

FIG. 1D is a top view of the bite block body of FIG. 1C, according to at least one embodiment of the present disclosure;

FIG. 1E is a perspective view of the bite block of FIG. 1A as used in connection with a nasal cannula, according to at least one embodiment of the present disclosure;

FIG. 1F is an exploded view of the bite block of FIG. 1A, according to at least one embodiment of the present disclosure;

FIG. 2 is a side view of a bite block inserted into the mouth of a patient, according to at least one embodiment of the present disclosure;

FIG. 3 is a perspective view of a bite block, according to at least one embodiment of the present disclosure;

FIG. 4 is a perspective view of a bite block, according to at least one embodiment of the present disclosure;

FIG. 5A is a front view of a bite block, according to at least one embodiment of the present disclosure;

FIG. 5B is a top view of the bite block of FIG. 5A, according to at least one embodiment of the present disclosure;

FIGS. 5C and 5D are side views of the bite block of FIG. 5A, according to at least one embodiment of the present disclosure.

FIG. 6 is a flowchart of a method of using a bite block, according to at least one embodiment of the present disclosure.

### DETAILED DESCRIPTION

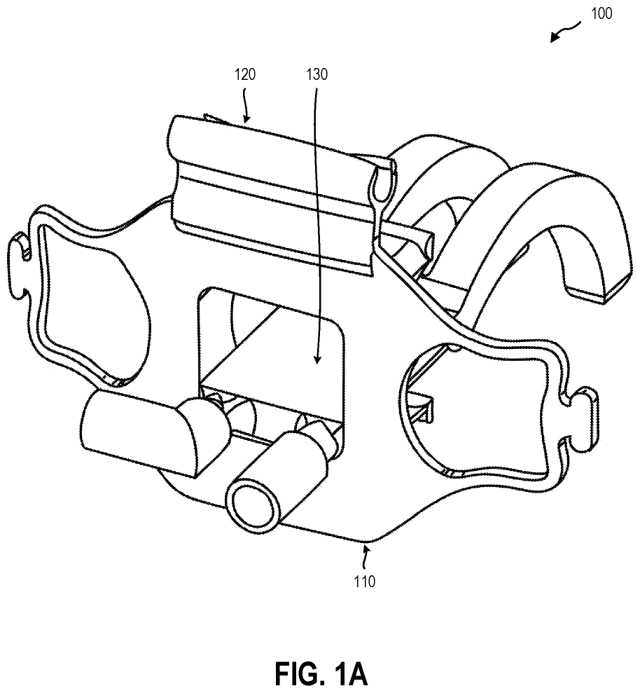

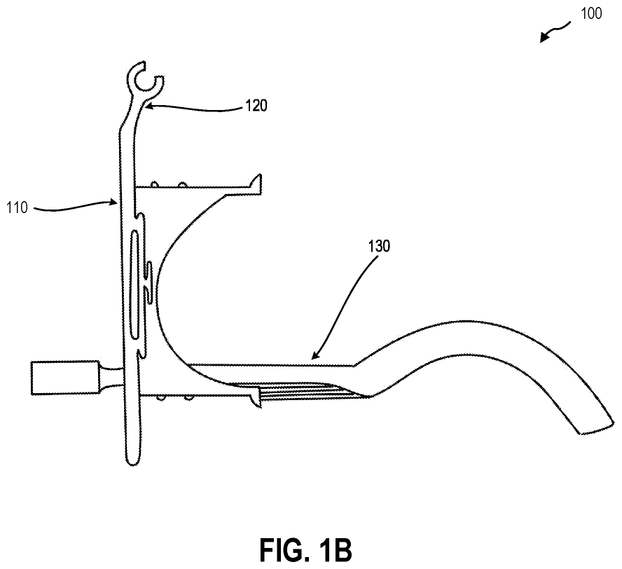

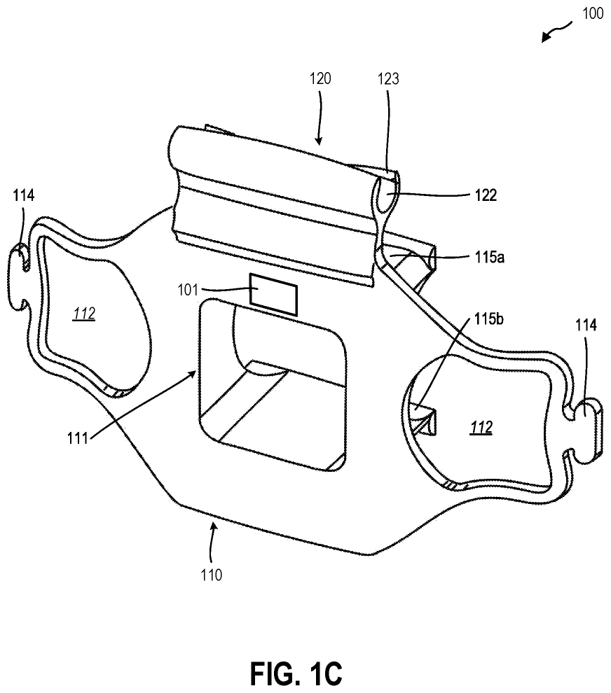

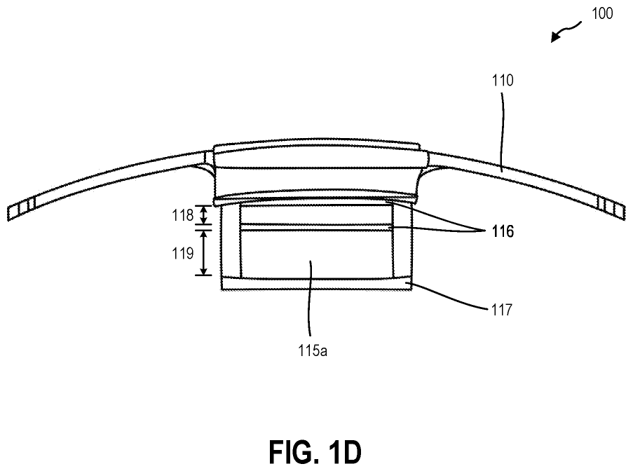

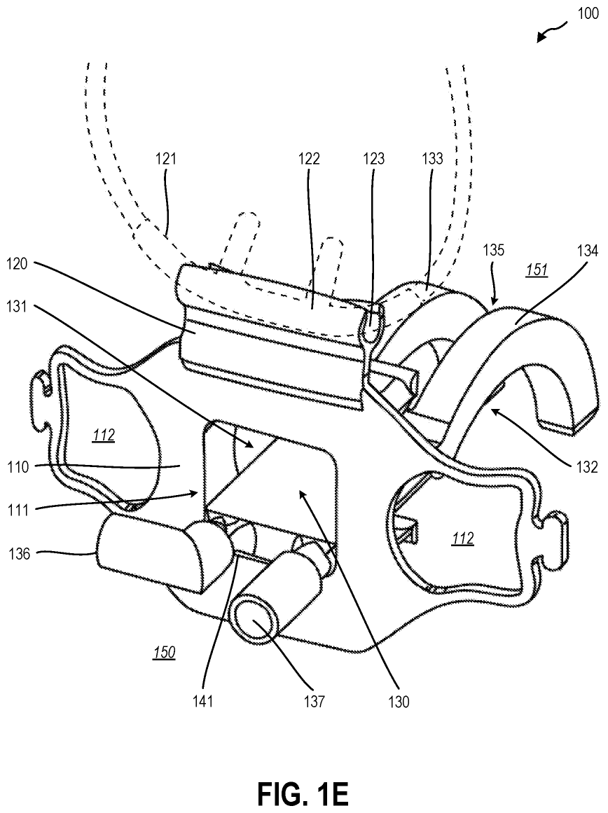

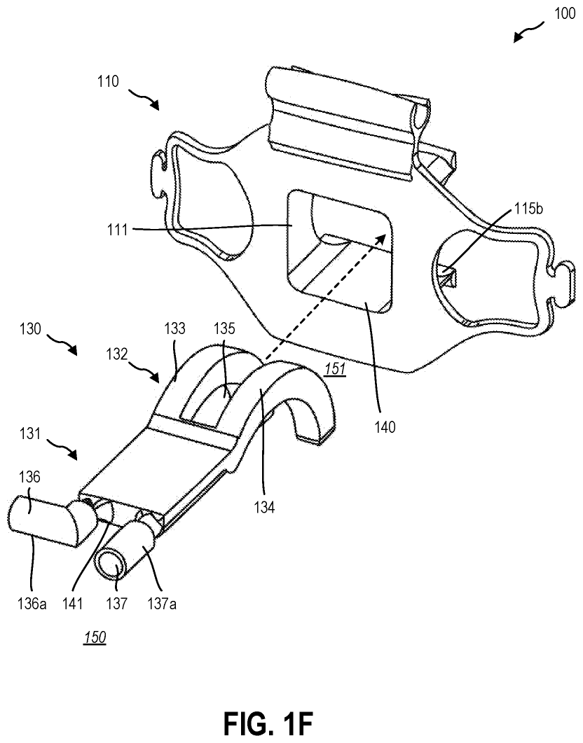

This disclosure generally relates to bite blocks and mouth guards used in endoscopic and other oral procedures. FIG. 1A is a representation of a perspective view of a bite block 100, according to at least one embodiment of the present disclosure. The bite block 100 may be configured to be inserted into a patient's mouth. Tools for various procedures may be inserted into the patient's mouth through the bite block 100. These procedures may include endoscopic procedures, endotracheal procedures, and other procedures in which an instrument or a tool is inserted into the patient's mouth. The present disclosure may discuss various features as being a probe opening or an endoscopy port. However, this should not be interpreted to limit the probe opening or endoscopy port to probes, endoscopic procedures, or other instruments and/or tools, but rather should be inclusive of applications of the bite block in other uses, for example, instruments and tools used in other procedures, such as endotracheal instruments and tools. The bite block 100 may protect areas of the patient's mouth, including the teeth, the tongue, the palate, the throat, and combinations of the foregoing, from damage by the instrument or tool inserted into his or her mouth. The bite block 100 may further protect the instruments and tools that are inserted into the patient's mouth from damage by the patient's teeth and other areas of the patient's mouth.

As shown in FIGS. 1A and 1B, the bite block 100 may include a bite block body 110 and an oral airway body 130. The bite block 100 may also include a nasal cannula holder 120. The bite block body 110, oral airway body 130, and nasal cannula holder 120 may be integrally formed as one piece or may be separately formed as two or more pieces that detachably connect to one another to form bite block 100. The nasal cannula holder may be located on an upper portion of the bite block 100. The oral airway body may be located on a lower portion of the bite block 100 and extend through the bite block body 110.

FIG. 1C is a representation of a perspective view of the bite block body 110 of the bite block 100 of FIG. 1A, according to one embodiment of the present disclosure. The bite block 100 may include a probe opening 111 that extends through a central portion of the bite block body 110. The probe opening 111 may be circular, square, rectangular, or any other shape. In one embodiment the probe opening 111 is a square and measures 24 mm by 24 mm. The probe opening 111 may be large enough to fit and maneuver a working portion of a medical instrument. In this way, when the bite block 100 is used in connection with a patient, the probe opening 111 allows for access around a medical instrument, such as a standard 16 mm endoscopic probe, when the medical instrument is used to access the oral cavity of a patient through the bite block 100. In some embodiments, the probe opening 111 may allow for extra access around the probe for additional instruments or other elements a health care practitioner may use to access the oral cavity of a patient, such as the practitioner's fingers or other medical instruments.

The bite block 100 shown in FIG. 1C includes one or more of auxiliary openings 112. The auxiliary openings 112 may be located on and pass through the bite block body 110. In some embodiments, the auxiliary openings 112 may measure 21.3 mm by 22.5 mm in order to allow access and sufficient clearance for fingers or additional medical instruments to access the oral cavity of a patient using the bite block 100. One or more of the auxiliary openings 112 may be located on a side of the probe opening 111. The auxiliary openings 112 may be located on the same side or on opposite sides of the probe opening 111. In this way, the auxiliary openings 112 may provide anterior oral access for use of a medical instrument such as a suction catheter or a physician's finger. Use of the medical instrument or physician's finger in this way may be supplementary, or in addition to use of a medical instrument through the probe opening 111. In some situations, the auxiliary openings 112 may provide anterior oral access for a physician's finger to help guide the insertion of an endoscopic probe. In some situations, the auxiliary openings 112 may provide anterior oral access for use of a rigid or soft suction catheter to remove any secretions as may be present in a patient's mouth or throat.

The auxiliary openings 112 may be large enough to fit and maneuver fingers, or medical instruments. In this way, when the bite block 100 is used in connection with a patient, the auxiliary openings 112 allow for access to the oral cavity of a patient through bite block 100.

The bite block 100 may include an upper biting platform 115a and a lower biting platform 115b. The upper biting platform 115a may be located on an upper portion of the bite block body 110. The lower biting platform 115b may be located on a lower portion of the bite block body 110. The upper biting platform 115a may be located above the probe opening 111 and the lower biting platform 115b may be located below the probe opening 111. When bite block 100 is used in connection with a patient, the patient's teeth may rest on the upper biting platform 115a and lower biting platform 115b. This may help to prevent the patient from closing their mouth or biting into the space defined by the probe opening 111. In this way, the bite block 100 may keep a patient's mouth open and prevent him or her from biting down on a medical instrument or a finger inserted into the patient's mouth. This may further help to protect the patient by preventing damage or injury to the patient's teeth, mouth, tongue, throat, gums, any other portion of the patient's mouth, and combinations thereof. In some embodiments, this may help to reduce or prevent damage to the physician and/or a medical instrument.

In some embodiments, the bite block 100 may be capable of supporting the stress of a patient biting down on the upper biting platform 115a and lower biting platform 115b. For this purpose, the upper biting platform 115a and lower biting platform 115b may have dimensions of 30 mm by 15 mm, and may have a material thickness of 3 mm. In this way, the upper biting platform 115a, the lower biting platform 115b, and the bite block 100 may support a constant stress of 1.08 MPa, representative of an average force that a patient may exert on the upper biting platform 115a and lower biting platform 115b during use. In some embodiments, the bite block 100 may support a constant stress of greater than 1.08 MPa. In some embodiments, the material thickness may be thicker or thinner than 3 mm depending on the material used in the construction of the bite block 100.

In some embodiments, the bite block 100 may be fabricated from a hard material, such as an extruded plastic, an injection molded plastic, a polymeric material, a hard rubber, a composite material, a metal, a metallic alloy, any other material, and combinations thereof. In some embodiments, the bite block 100 may include a soft material located on the upper biting platform 115a and/or the lower biting platform 115b. The soft material may be incorporated in the upper biting platform 115a and/or the lower biting platform 115b in the form of a soft insert on the upper biting platform 115a and/or the lower biting platform 115b. The upper biting platform 115a and/or the lower biting platform 115b may also be entirely composed of a soft material. In this way, the teeth of a patient using the bite block 100 may be cushioned against the hard material of the bite block 100.

The bite block 100 may also include tabs 114 connected to an outer portion of the bite block body 110. The tabs 114 may be generally “T-shaped”. A securing strap may connect to the tabs 114 and wrap around a patients head to secure the bite block 100 in place, and in this way maintain the bite block 100 in place. The tabs 114 and the securing strap may also prevent the bite block 100 from entering too far into a patient's mouth and thus may prevent the bite block 100 from becoming a choking hazard.

The bite block 100 may include a nasal cannula holder 120. In connection with the use of the bite block 100 with a patient, the nasal cannula holder 120 may be configured to accept and retain a nasal cannula in a position relative to the nose of a patient and allow a flow of oxygen to the patient's nose in substantially the same way as would a nasal cannula without the use of the bite block 100. For example, when the patient has inserted the bite block 100 into his or her mouth, a nasal cannula secured by the nasal cannula holder 120 may be located in a manner to allow the openings of the nasal cannula to be inserted into the patient's nostrils. Additionally, the nasal cannula holder may retain the nasal cannula without impeding the functionality of the nasal cannula. Put another way, the nasal cannula holder may hold the nasal cannula without impeding the normal flow of oxygen through the nasal cannula. In this way, the bite block may be used in conjunction with, and facilitate the use of a nasal cannula for providing medical oxygen to a patient.

Conventionally, during an operation, such as an endoscopic operation, a nasal cannula may fully or partially be removed from a patient's nostrils. For example, movement of the patient's head may jostle, bump, or otherwise move the nasal cannula. In some examples, a health care provider or other person in the procedure room may bump the bite block 100, the nasal cannula, and/or the oxygen tubes connected to the nasal cannula. This may cause one or more of the openings of the nasal canula to be removed from the patient's nostrils, thereby reducing the oxygen flow to the patient's lungs. In accordance with at least one embodiment of the present disclosure, the nasal cannula holder 120 may help to secure the nasal cannula to the user's nostrils during an operation, such as an endoscopic operation. Because the nasal cannula is fixed to the nasal cannula holder 120, and the patient has secured the bite block 100 to his or her mouth by biting on the upper biting platform 115a and/or the lower biting platform 115b, the nasal cannula may remain fixed in place relative to the patient's nose. This may help to reduce or prevent the nasal cannula from being removed from the patient's nostrils, thereby maintaining the oxygen flow rate to the patient.

In accordance with at least one embodiment of the present disclosure, the bite block 100 may include a carbon dioxide sensor 101. The carbon dioxide sensor 101 may be located at a location to detect the carbon dioxide content of the patient's exhalations. This may allow the health care provider to determine whether the patient is properly respirating. The carbon dioxide sensor 101 may be located at any location. For example, the carbon dioxide sensor 101 may be located at the probe opening 111. In some examples, the carbon dioxide sensor 101 may be located at the nasal cannula holder 120. In some examples, the carbon dioxide sensor 101 may be located and/or a part of the nasal cannula. In some examples, the carbon dioxide sensor 101 may be located inside the patient's mouth, such as in a probe or other instrument. In some examples, the carbon dioxide sensor 101 may be located at the back of the user's throat, such as connected to the suction device and/or a portion of the probe. Including the carbon dioxide sensor 101 in the bite block 100 may allow the healthcare provider to monitor the patient's respiration and potentially make changes to the patient's care based on the monitored carbon dioxide levels.

In the embodiment shown, the nasal cannula holder 120 includes a nasal cannula channel 122 and a nasal cannula channel opening 123. The nasal cannula may be inserted into the nasal cannula channel 122 through the nasal cannula channel opening 123. The nasal cannula channel opening 123 may grip the body of the nasal cannula to secure the nasal cannula to the bite block 100. However, it should be understood that the nasal cannula holder 120 may include any mechanism to secure a nasal cannula to the bite block 100. In some embodiments, the nasal cannula holder 120 may be integrally formed with the bite block body 110. This may help to prevent the nasal cannula holder 120 from moving with respect to the bite block body 110, which may help to maintain positioning of the nasal cannula in the patient's nostrils.

FIG. 1D shows a top view of the bite block 100 of FIG. 1C. As shown, the bite block body 110 may have a generally curved shape. In this way, when inserted into the mouth of a patient, the bite block body 110 may generally fit to the form of the patient's mouth, lips and/or face. The bite block 100 may include one or more retainers 117. One or more retainers 117 may be located on an upper face of the upper biting platform 115a, a lower face of the lower biting platform 115b, or both. The retainer 117 may be located on a posterior edge of the upper biting platform 115a and/or the lower biting platform 115b and may help to prevent the bite block 100 from sliding out of a patient's mouth when in use.

In some embodiments, the retainer 117 may include a ridge, protrusion or other element that extends upward from a surface of the upper biting platform 115a and/or the lower biting platform 115b In this manner, when the patient bites down on the upper biting platform 115a and the lower biting platform 115b and an outward force is applied to the bite block 100, the retainer 117 may contact the user's teeth and prevent removal of the bite block 100 from the patient's mouth.

The bite block 100 may include one or more ribs 116. The ribs 116 may be located on an upper face of the upper biting platform 115a, on a lower face of the lower biting platform 115b, or both. The ribs 116 may be separated by a first distance 118. The first distance 118 may be 3 mm. The ribs 116 may also be separated from the retainer 117 by a second distance 119. The second distance 119 may be 7.45 mm as measured from the retainer 117 to the nearest rib.

The ribs 116 may serve to keep the bite block in place while in use in the mouth of a patient. This may be achieved by the patient biting down on the upper biting platform 115a and lower biting platform 115b between the ribs 116. This may also be achieved by the patient biting down on the upper biting platform 115a and lower biting platform 115b between the retainer 117 and the nearest rib. In this way, the ribs 116 and/or retainer 117 prevent the bite block 100 from sliding too far forward or backward within the mouth of a patient. The ribs 116 and/or retainer 117 may also serve to stabilize the bite block 100 against movement of the bite block 100 relative to the mouth of the patient due to manipulations of medical instruments through the bite block 100, movement of the patient, etc.

FIG. 1E is a representation of a perspective view of the bite block 100 of FIG. 1A connected to the nasal cannula 121, in accordance with at least one embodiment of the present disclosure. The bite block 100 shown includes a nasal cannula holder 120 including a nasal cannula channel 122. The nasal cannula channel 122 may be relatively cylindrical in shape. The width or diameter of the nasal cannula channel 122 may be 5.5 mm, representative of the width of a typical nasal cannula 121. The shape of the nasal cannula channel 122 may be representative of only a partial cylinder, and may further be defined by a nasal cannula channel opening 123 located along a partial circumference of the nasal cannula channel 122. The nasal cannula channel opening 123 may extend an entire length of the nasal cannula channel 122 and may have a width less than the width or diameter of the nasal cannula channel 122 such that the nasal cannula channel 122 has a relatively “c-shaped” profile. The nasal cannula channel opening 123 may be located along a portion of the circumference of the nasal cannula channel 122 that is at a substantially vertical point, or on the top of the nasal cannula channel 122. The nasal cannula channel opening may also be located along a portion of the circumference of the nasal cannula channel 122 that is angled away from vertical relative to the nasal cannula channel 122.

The nasal cannula holder 120 may be capable of accepting a nasal cannula 121 through the nasal cannula channel opening 123 and into the nasal cannula channel 122. In this way, a nasal cannula 121 may be press fit into the nasal cannula channel 122 by relying on the flexible nature of a typical nasal cannula 121 to press through the nasal cannula channel opening 123. Nasal cannula 121 as described in this disclosure may be any type of nasal cannula used to deliver medical oxygen to the nostrils including a standard nasal cannula, nasal cannula with EtC02 monitoring, or a high-flow nasal cannula. The nasal cannula 121 may be a curved prong cannula, flared prong cannula, curved/flared prong cannula, straight prong cannula, or any other nasal cannula. In some embodiments, the nasal cannula channel 122 may have a diameter or width that is more or less than 5.5 mm to accept various sizes of nasal cannula 121.

The nasal cannula holder 120 may be located on an upper portion of a bite block body 110 and may extend upwardly from the bite block body 110 (e.g., away from the probe opening 111 and toward a nose of the patient). The nasal cannula holder 120 may be angled toward the nose of the patient when the bite block 100 is being held in the patient's mouth. The nasal cannula holder 120 may be positioned such that it may hold the nasal cannula 121 in an appropriate position relative to the nose of a patient such that the nasal cannula 121 will function and deliver nasal oxygen to a patient in substantially the same way as it would through typical use of a nasal cannula 121 without use of the bite block 100. The distance that the nasal cannula holder 120 extends upwardly may be 13.85 mm, and the angle of the nasal cannula holder 120 toward the patient's nose may be 30° from vertical, representing a position of the nasal cannula holder corresponding to the average physiology of a patient using the bite block 100.

The bite block 100 shown includes an oral airway body 130 connected to the bite block body 110. In some embodiments, the oral airway body 130 may be removably connected to the bite block body 110. The oral airway body 130 may be inserted into the probe opening 111.

The oral airway body 130 may include an anterior portion 131 and a posterior portion 132. A connecting surface 141 may be located on the anterior portion 131. In this manner, the anterior portion 131 may serve, at least in part, to connect the oral airway body 130 to the bite block body 110. The anterior portion 131 may be generally flat in shape. The posterior portion 132 may comprise a first portion 133 and a second portion 134. The first portion 133 and the second portion 134 may be separated by a gap 135 such that the posterior portion 132 has a split or a forked shape.

The posterior portion 132 may have a generally curved shape, and the curved shape may curve in a downward direction. When the bite block 100 is inserted into the patient's mouth, the posterior portion 132 may extend to the back of the patient's mouth and pull the tongue down and forward. This may help to open up the mouth passage of the patient during a procedure. The curved shape of the posterior portion 132 may rise to a distance above a horizontal axis of the anterior portion 131 before curving in a downward direction a distance below a horizontal axis of the anterior portion 131. In this way, when the oral airway body 130 is inserted into the mouth of a patient, the posterior portion 132 may rest on the tongue of the patient. In some embodiments, some or all of the anterior portion 131 may rest on the tongue of the patient. When inserted into the mouth of a patient, an end of the first portion 133 and of the second portion 134 may be located at the base of the patient's tongue, and/or the posterior oropharynx. In this way, at least a portion of the oral airway body 130 ends at the base of the patient's tongue allowing for posterior oral access while also acting as an oral airway to pull the tongue forward.

During an endoscopic procedure, a healthcare provider may insert a medical instrument into the mouth or throat of a patient through the probe opening 111. In some embodiments, the gap 135 may serve as a guide for the insertion of a probe, endoscope, or other medical device to the center of the throat of the patient. For example, when the oral airway body 130 is inserted into the mouth of a patient, the gap 135 may help to guide an endoscope to an appropriate location during oral application of the endoscope. This may serve to ease the insertion of the endoscope or other medical device as well as to prevent or reduce damage to the patient's throat or other tissues.

The oral airway body 130 may include a first fluid passage 136 and a second fluid passage 137. The first fluid passage 136 and the second fluid passage 137 may each have a proximal end at a front 150 of the oral airway body 130 and a distal end at a back 151 of the oral airway body 130. The first fluid passage 136 may be hydraulically or fluidly connected between the front 150 and the back 151 of the oral airway body 130 and the second fluid passage 137 may be hydraulically or fluidly connected between the front 150 and the back 151 of the oral airway body 130. Put another way, the first fluid passage 136 and the second fluid passage 137 may each include a passage or chamber that passes through the oral airway body 130 by passing through the anterior and posterior portions 131 and 132. The first fluid passage 136 may pass through the first portion 133 and the second fluid passage 137 may pass through the second portion 134. In this way, the first fluid passage 136 may define a first fluid path between the proximal and distal ends of the first fluid passage 136, and the second fluid passage 137 may define a second fluid path between the proximal and distal ends of the second fluid passage 137.

In some embodiments, the first fluid passage 136 may be an oxygen passage, and the first fluid path may be an oxygen fluid path. A flow of oxygen may be passed through the first fluid passage 136 to the back 151 of the oral airway body 130. The flow of oxygen may be an oral oxygen flow. The flow of oxygen through the first fluid passage 136 may be high-flow oxygen, and the first fluid path may be a high-flow oxygen fluid path. For example, the flow of oxygen through the first fluid passage 136 may be greater than 10 L/min, greater than 12 L/min, greater than 15 L/min, or any value therebetween.

In some embodiments, and as discussed herein, an end of the first portion 133 may rest at the base of the tongue or at the posterior oropharynx of a patient when the oral airway body 130 is inserted into the mouth of the patient. Oxygen passed through the first fluid passage 136 may be delivered directly to the posterior oropharynx of the patient. This may help to provide oxygen to a patient during an endoscopic operation. Oxygen provided directly to the airway of a patient may be more effective at maintaining an appropriate blood oxygenation level. In this way, providing oxygen through the first fluid passage may also help to more effectively provide oxygen to a patient than by providing nasal oxygen to a patient such as through the nasal cannula 121.

In some embodiments, during a procedure, the first fluid passage 136 and the nasal cannula 121 may both provide oxygen to the patient. In some embodiments, the first fluid passage 136 may provide oxygen to the patient simultaneously with the nasal cannula 121. Put another way, both the nasal cannula 121 and the first fluid passage 136 may provide oxygen to the patient during an operation. This may help to increase the oxygen flow to the patient. In some embodiments, the first fluid passage 136 and the nasal cannula 121 may provide oxygen to the patient at different times during the procedure. For example, different portions of the procedure may include different oxygenation requirements and/or different oxygen uptake mechanisms. By including both a first fluid passage 136 and a nasal cannula 121, the healthcare provider may tailor the oxygen flow to the patient based on the operation being performed.

In some embodiments, the oxygen flow through the first fluid passage 136 may be supplementary to, or in addition to an oxygen flow delivered through the nasal cannula 121. In some embodiments, the oxygen flow through the first fluid passage 136 may be delivered from a supply of oxygen or through a fluid path that is distinct from an oxygen flow delivered through the nasal cannula 121. Put another way, the first fluid path defined by the first fluid passage 136 may be separate from a fluid path of the nasal cannula 121. This may allow the first fluid passage 136 to be connected to a separate oxygen source (such as separate wall plugs, or separate oxygen tanks) from the nasal cannula 121. Put another way, the oxygen flow from the first fluid passage 136 may not be connected to the oxygen flow from the nasal cannula 121. In this manner, the total volume of oxygen provided to the patient may be increased and/or the supply may be uninterrupted.

In some embodiments, the first fluid passage 136 at the front 150 of the oral airway body 130 may be situated at an angle relative to the oral airway body 130. This may help to redirect oxygen tubes and other elements away from the probe opening 111 of the bite block 100. This may help to prevent the primary operating field from becoming too cluttered. This may also serve as an additional securing mechanism that prevents the oral airway body 130, the bite block body 110, and/or the bite block 100 from sliding back into the patient's mouth and becoming a choking hazard.

In some embodiments, the second fluid passage 137 may be a suction passage and the second fluid path may be a suction fluid path. The second fluid passage 137 may provide suction to the oropharynx of the patient. In this way, a suction flow may pass through the second fluid passage 137 to the throat of the patient. As described herein, an end of the second portion 134 (which is fluidly connected to the second fluid passage 137) rests at the base of the tongue or at the posterior oropharynx of a patient when the oral airway body 130 is inserted into the mouth of the patient. In this way, the second fluid passage 137 may provide a suction flow directly to the posterior oropharynx of the patient and may remove secretions that lie in the oropharynx. This may serve to prevent complications that may trigger a laryngospasm, asphyxiation, or other medical condition during an oral procedure.

In some embodiments, the second fluid passage 137 may provide suction to the posterior oropharynx independently from the oxygen flow of the first fluid passage 136. In some embodiments, the second fluid passage 137 may provide suction simultaneously to the oxygen flow in the first fluid passage 136, or when the first fluid passage 136 is providing oxygen to the patient. In some embodiments, the second fluid passage 137 may provide suction at the same time that oxygen is supplied through the first fluid passage 136. In some embodiments, the second fluid passage 137 may provide suction simultaneously to one or more additional suction devices, such as a soft suction catheter, inserted through one or more of the auxiliary openings 112 or the probe opening 111.

In some embodiments, the second fluid passage 137 may be connected to a suction vacuum. In this manner, fluid may be directly sucked in through the second portion 134 at the back 151 of the oral airway body 130 and out of the opening of the second fluid passage 137. In some embodiments, the second fluid passage 137 may receive a soft suction tube. The soft suction tube may be inserted into the second fluid passage 137, through the second fluid passage 137, and out of the back 151 of the second portion 134. This may allow the healthcare provider to direct the suction to a desired location.

In some embodiments, the second fluid passage 137 may provide an oral oxygen flow to the patient and the second fluid path may be an oxygen fluid path. In some embodiments, the second fluid passage 137 may provide oxygen to the patient simultaneously with the first fluid passage 136. This may help to increase the oxygen flow at the posterior oropharynx.

FIG. 1F is a representation of an exploded view of the bite block 100 of FIG. 1A, according to an embodiment of the present disclosure. The bite block 100 may include a bite block body 110 and an oral airway body 130. In some embodiments, the oral airway body 130 may be connected to the bite block body 110. In some embodiments, the oral airway body 130 and the bite block body 110 may be integrally formed as one piece or may be separately formed as two or more pieces. The oral airway body 130 may detachably connect to the bite block body 110. In this way, the bite block 100 may be composed of one or more pieces that may be assembled before or during use of the bite block 100. This may aid in the manufacturing of the bite block 100. In some embodiments, a separately formed bite block body 110 and oral airway body 130 may allow the bite block body 110 and the oral airway body 130 to be used individually.

The bite block body 110 may have a probe opening 111. The probe opening 111 may include a receiving surface 140 located on a lower portion of the probe opening 111. The receiving surface 140 may be an upper surface of a lower biting platform 115b. The oral airway body 130 may be connected to the bite block body 110 at the receiving surface 140 of the probe opening 111. To connect to the oral airway body 130, the receiving surface 140 may have one or more chamfers on one or more outer edges of the receiving surface 140. The oral airway body 130 may have a connecting surface 141 located on a lower portion of the oral airway body. The connecting surface 141 may have one or more rounded edges corresponding to one or more chamfers on the receiving surface 140. In this way, the connecting surface may have a shape and/or dimensions that are substantially the same as those of the receiving surface 140. In this way, the oral airway body 130 may connect to the bite block body 110 through a connection of the connecting surface 141 with the receiving surface 140. The connection between the connecting surface 141 and the receiving surface 140 may be a press fit, a snap fit, an interference fit, or any other connection. The connecting surface 141 and the receiving surface 140 may each contain one or more additional features that may serve to achieve a connection between the surfaces and between the bite block body 110 and the oral airway body 130. These additional features may amount to a connection such as a snapping connection, a connection with a fastener, an adhesive connection, or any other suitable means for connecting the bite block body 110 and the oral airway body 130.

In some situations, an operation or a portion of an operation may utilize only the bite block body 110. The healthcare professional may insert only the bite block body 110 into the mouth of a patient. In some embodiments, a second portion of the operation may utilize the oral airway body 130 in conjunction with the bite block body 110. The healthcare provider may insert and/or connect the oral airway body 130 to the bite block body 110. For this purpose, the width of the oral airway body 130 may be less than the width of a probe opening 111 such that the oral airway body 130 may fit and/or pass partially or entirely through the bite block body 110 via the probe opening 111. Put another way, an oral airway width of the oral airway body 130 may be less than a probe opening width of the probe opening 111 such that the oral airway body 130 may fit and/or pass through the probe opening 111. In this way, the bite block body 110 and the oral airway body 130 may be connected by inserting the oral airway body 130 into and partially through the probe opening 111. In some embodiments, the oral airway body 130 may be inserted into the probe opening 111 from either side of the probe opening 111.

The oral airway body 130 may include an anterior portion 131 and a posterior portion 132. The connecting surface 141 may be located on the anterior portion 131, and therefore the anterior portion 131 may serve, at least in part, to connect the oral airway body 130 to the bite block body 110. The anterior portion 131 may be generally flat in shape. The posterior portion 132 may comprise a first portion 133 and a second portion 134. The first portion 133 and the second portion 134 may be separated by a gap 135 such that the posterior portion 132 has a split or a relatively forked shape.

In some situations, an oral device may extend to the back of a patient's tongue in order to pull the tongue forward. For this purpose, the posterior portion 132 may have a generally curved shape, and the curved shape may curve in a downward direction. The curved shape of the posterior portion 132 may also rise to a distance above a horizontal axis of the anterior portion 131 before curving in a downward direction a distance below a horizontal axis of the anterior portion 131. In this way, when the oral airway body 130 is inserted into the mouth of a patient, the posterior portion 132 may rest on the tongue of the patient. Additionally, some or all of the anterior portion 131 may rest on the tongue of the patient. When inserted into the mouth of a patient, an end of the first portion 133 and of the second portion 134 may be located at the base of the patient's tongue, and/or the posterior oropharynx. In this way, at least a portion of the oral airway body 130 ends at the base of the patient's tongue allowing for posterior oral access while also acting as an oral airway to pull the tongue forward.

The oral airway body 130 may include a first fluid passage 136 and a second fluid passage 137. The first fluid passage 136 and the second fluid passage 137 may each have a proximal end at a front 150 of the oral airway body 130 and a distal end at a back 151 of the oral airway body 130. In this way, the first fluid passage 136 and the second fluid passage 137 may pass through the oral airway body 130 by passing through the anterior and posterior portions 131 and 132. The first fluid passage 136 may pass through the first portion 133 and the second fluid passage 137 may pass through the second portion 134.

The first fluid passage 136 may include a first connector 136a. The first connector 136a may be an oxygen adapter configured to connect to a supply of medical oxygen. The first connector 136a may be any type of oxygen adapter. For example, the first connector 136a may be a standard barbed oxygen adaptor (i.e., a barbed male end that connects to tubing with a 5 mm-7 mm internal diameter), a swivel oxygen adaptor (i.e., a barbed male end having a middle portion that swivels for less tubing issues such as swivel oxygen tubing connectors offered by Salter Labs or WestMed), or any other oxygen adaptor that is suited to connect to a 5 mm-7 mm female tubing. In this way, a flow of oxygen may pass through the first fluid passage 136.

The first connector 136a may be situated at an angle relative to the oral airway body 130. In one embodiment, the first connector 136a is angled at 90° away from the second fluid passage. In this way, the first connector 136a is angled away from the probe opening 111 when the oral airway body 130 is connected to the bite block body 110. This may serve to prevent the primary operating field from becoming too cluttered. This may also serve as an additional securing mechanism that prevents the oral airway body 130, the bite block body 110, and/or the bite block 100 from sliding back into the patient's mouth and becoming a choking hazard.

The second fluid passage 137 may include a second connector 137a. The second connector 137a may be a suction adapter configured to connect to a medical suction device such as a small, soft suction catheter. The second connector 137a may be a suction adaptor configured to connect to a 10 Fr soft suction catheter. The second connector 137a may be any type of suction adapter such as a suction adaptor configured to connect to a 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, or 18 Fr soft suction catheter or any other medical suction device. In this way, a suction flow may pass through the second fluid passage 137.

As described above, an end of the second portion 134 may rest at the base of the tongue or at the posterior oropharynx of a patient when the oral airway body 130 is inserted into the mouth of the patient. In this way, the second fluid passage 137 may provide a suction flow directly from the posterior oropharynx of the patient and may remove secretions that lie in the oropharynx. This may serve to prevent complications that may trigger a laryngospasm, asphyxiation, or other medical condition during an oral procedure. The suction flow through the second fluid passage 137 may be supplementary to, or in addition to an oxygen flow delivered through the first fluid passage 136.

The second connector 137a may also be, or be replaceable with, an oxygen adapter such as that described in connection with the first connector 136a. In this way, the second fluid passage 137 may serve as a suction passage providing generalized static suction through a standard oxygen adapter such as those described herein in connection with the first connector 136a. The second fluid passage 137 may also serve as an oxygen passage as described in connection with the first fluid passage 136, and a flow of oxygen may pass through the second fluid passage 137. This flow of oxygen may be supplementary to, or in addition to the flow of oxygen through the first fluid passage 136. Additionally, this flow of oxygen may be supplementary to, or in addition to the flow of oxygen through the nasal cannula 121 as described above in conjunction to FIG. 1E. The oxygen flow through the second fluid passage 137 may be delivered from a supply of oxygen or through a fluid path that is distinct from the oxygen flow through the first fluid passage 136, and from the oxygen flow delivered through the nasal cannula 121 described above in conjunction with FIG. 1E.

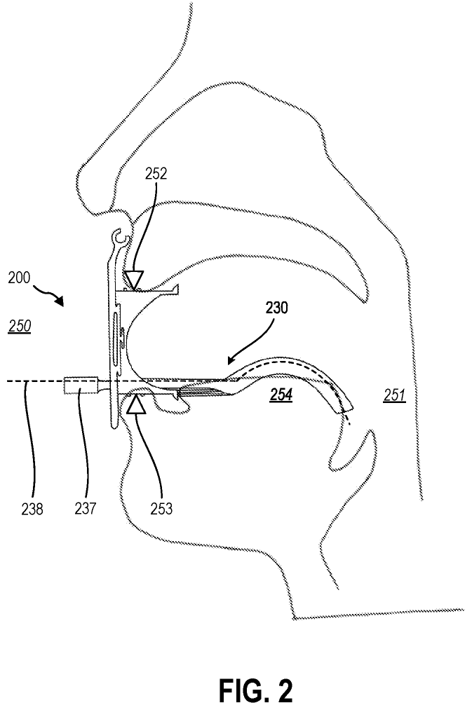

FIG. 2 is a representation of a side view of a bite block 200 inserted into the mouth of a patient, according to at least one embodiment of the present disclosure. When inserted, the bite block 200 may rest between the upper teeth 252 and lower teeth 253 of the patient and as such be held in place. The bite block 200 may include an oral airway body 230. When inserted, the oral airway body 230 may extend posteriorly into the patient's mouth such that the oral airway body 230 rests on and ends at the base of the patient's tongue 254. In this way, the oral airway body 230 allows for posterior oral access while also acting as an oral airway to pull the tongue 254 forward.

The oral airway body 230 may include a first fluid passage 236 and a second fluid passage 237 adjacent to the first fluid passage 236. The first fluid passage 236 may define a first fluid path 238 and the second fluid passage 237 may define a second fluid path. The first fluid path 238 may be an oxygen path as described above in conjunction with the first fluid passage 136 in FIG. 1F. The second fluid path may be an oxygen path or a suction path as described above in conjunction with the second fluid passage 137 in FIG. 1F. When the bite block 200 is inserted into the mouth of a patient, the first fluid path 238 and the second fluid path have a first end at a front 250 of the oral airway body 230 and a second end at a back 251 of the oral airway body 230. When inserted into the mouth of a patient, the back 251 of the oral airway body 230 may be located at the top of the airway, or at the posterior oropharynx of the patient. In this way, the first fluid passage 236 may deliver a flow of oxygen along the first fluid path 238 and directly to the airway of the patient. The second fluid passage may provide a suction flow along the second fluid path directly from the airway of the patient. The second fluid passage may also deliver a flow of oxygen along the second fluid path and directly to the airway of the patient.

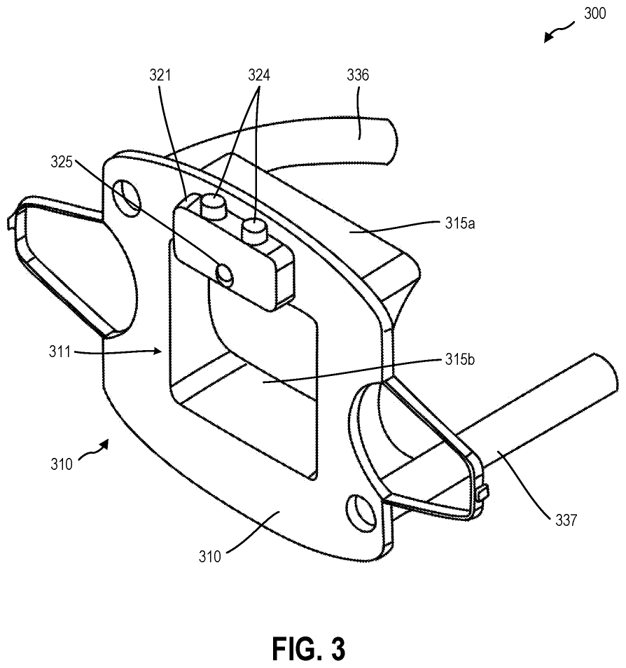

FIG. 3 is a representation of a perspective view of a bite block 300, according to at least one embodiment of the present disclosure. The bite block 300 may include a base 310, an upper biting platform 315a and a lower biting platform 315b connected to the base 310. The bite block 300 may include a probe opening 311 through the base 310. The probe opening 311 may be located between the upper and lower biting platforms 315a,b. The probe opening 311 may serve to allow oral access for medical instruments through the bite block 300.

The bite block may include a first fluid passage 336. The first fluid passage 336 may extend posteriorly from the base 310 and may have a generally curved shape. In this way, when the bite block 300 is used in connection with a patient, the first fluid passage 336 may extend to a posterior portion of the patient's mouth. The first fluid passage 336 may be an oxygen passage. The first fluid passage 336 may have an opening on the base 310 such that medical oxygen may be connected to the first fluid passage 336. In this way, a flow of oxygen may be delivered to the patient through the first fluid passage 336. The flow of oxygen may be delivered to a posterior portion of the patient's mouth. The flow of oxygen may be high-flow oxygen of 10 L/min or more. The first fluid passage 336 may be located on an upper portion of the bite block 300. In this way, when the bite block 300 is inserted into the mouth of a patient, the first fluid passage 336 may be located adjacent to the roof of the patient's mouth.

The bite block may include a second fluid passage 337. The second fluid passage 337 may extend posteriorly from the base 310. In this way, when the bite block 300 is used in connection with a patient, the second fluid passage 337 may extend to a posterior portion of the patient's mouth. The second fluid passage 337 may be a suction passage. The second fluid passage 337 may have an opening on the base 310 such that a medical suction device may be connected to the second fluid passage 337, such as a soft suction catheter. In this way, a suction flow may be provided through the second fluid passage 337. A suction flow may be provided through the second fluid passage 337 from a posterior portion of the patient mouth such that secretions may be removed directly from the patient's airway. The second fluid passage may be located on a lower portion of the bite block 300. In this way, when the bite block 300 is inserted into a patient's mouth, the second fluid passage 337 may be located adjacent to the patient's tongue.

The first fluid passage 336 and the second fluid passage 337 may be located on opposite sides of the probe opening 311. The first fluid passage 336 and the second fluid passage 337 may be located on opposite portions (upper and lower) of the bite block 300. This may provide stability for the bite block 300 against movement when it is inserted into the mouth of a patient. The first fluid passage 336 and the second fluid passage 337 may be located in substantially opposite positions on the bite block 300. In this way, the first fluid passage 336 and the second fluid passage 337 do not block access to the patient's mouth through the probe opening 311.

The bite block 300 may also include a nasal cannula 321. The nasal cannula 321 may be located on a front face of the base 310, and on an upper portion of the base 310. The nasal cannula may include two nasal passages 324 extending from a top of the nasal cannula 321. The nasal passages 324 may extend into the nostrils of a patient when the bite block 300 is inserted into the mouth of the patient. The nasal cannula 321 may also include an oxygen opening 325. The oxygen opening 325 may be located on a front of the nasal cannula 321. The oxygen opening 325 may be connected to the nasal passages 324 such that a fluid path passes through the oxygen opening 325 and through one or more of the nasal passages 324. The oxygen opening 325 may connect to a medical oxygen device such that a flow of oxygen may pass through the nasal cannula 321. In this way, a flow of oxygen may be delivered to the nostrils of a patient through the nasal cannula 321 when the bite block 300 is inserted into the patient's mouth.

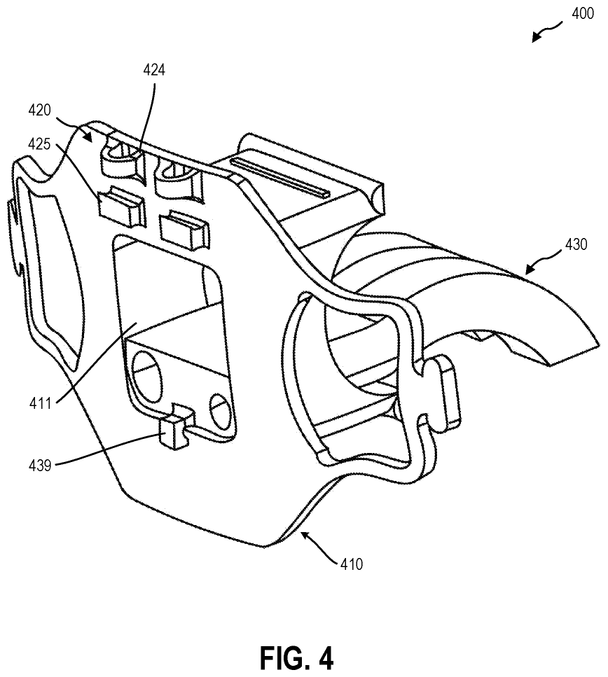

FIG. 4 shows a perspective view of a bite block 400 according to one embodiment of the present disclosure. The bite block 400 may include a base 410, a probe opening 411, and an oral airway body 430. The oral airway body 430 may extend posteriorly from the base 410 and through the probe opening 411. The oral airway body may be detachably connected to the base 410. The oral airway body may include a clip 439. The clip 439 may connect to the base 410 at a lower portion of the probe opening 411. The base 410 may include a slot located below the probe opening configured to receive the clip 439. In this way, the oral airway body 430 may detachably connect to the base 410 be securing the clip 439 to the base 410. The oral airway body 430 may be detachably connected to the base 410 after the base 410 is inserted into the mouth of a patient. In this way, the base 410 may be used separately from the oral airway body 430, and the oral airway body 430 may be inserted into the mouth of a patient through the probe opening 411 and secured to the base 410 via the clip 439.

The bite block 400 may also include a nasal cannula holder 420 located on an upper portion of the base 410. The nasal cannula holder 420 may include one or more hooks 424 and one or more holding platforms 425 located underneath the hooks 424. The hooks 424 may be configured to receive one or more nasal tubes of a nasal cannula, and the holding platforms 425 may be configured to receive a base portion of a nasal cannula. The holding platforms 425 may include a lip on an outer edge of the holding platforms 425 such that when a nasal cannula is inserted into the hooks 424 and onto the holding platforms 425, the nasal cannula will be held in place relative to the bite block 400. The holding platforms 425 may be positioned relative to the hooks 424 such that a nasal cannula may flex in order to fit past the lip and onto the holding platforms 425. In this way, a nasal cannula may be secured to the bite block 400. The nasal cannula holder 420 may be positioned on the base 410 such that, when the bite block is used in connection with a patient, a nasal cannula attached to the bite block 400 via the nasal cannula holder 420 will be positioned under the patient's nose and/or in the patient's nostrils in substantially the same way as would the nasal cannula without use of the bite block 400.

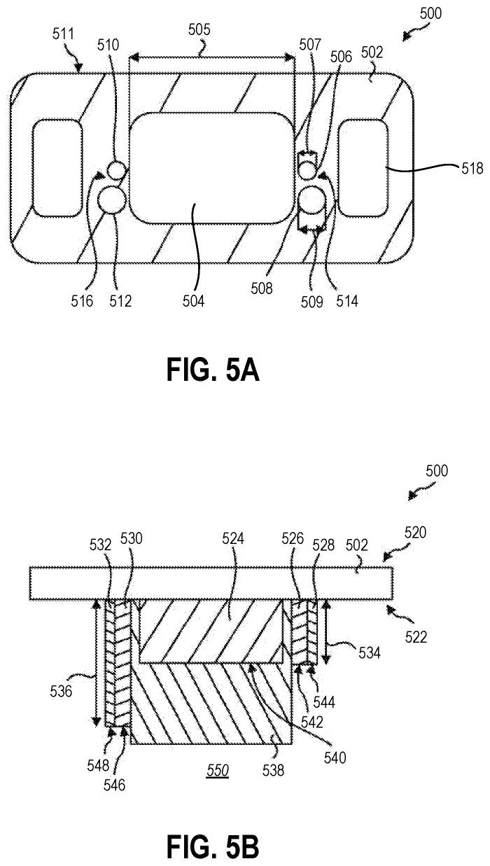

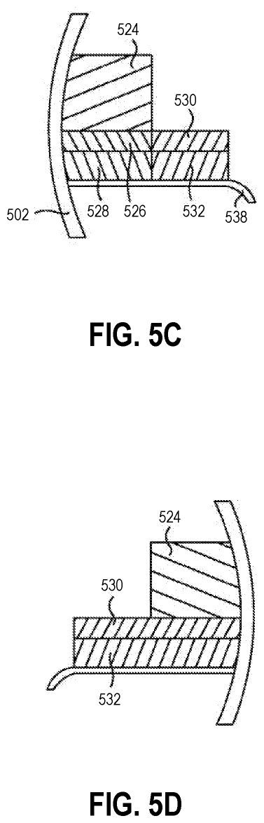

FIG. 5A is a front view of a bite block 500 according to one embodiment of the present disclosure. The bite block 500 may include a body 502. The body 502 may include an endoscopy port 504. In at least one embodiment, the endoscopy port 504 may be located in a center of the body 502, and thus may be a central port. In other embodiments, the endoscopy port 504 may not be located in the center of the body 502. For example, the endoscopy port 504 may be located above, below, left, or right of the center of the body 502. The endoscopy port 504 may be sized such that an endoscopy tool may be inserted through the endoscopy port 504 and into the patient's mouth. For example, the endoscopy port 504 may have an endoscopy port diameter 505 that is between 5 mm and 15 mm in diameter. For example, the bite block 500 of the present disclosure may be manufactured in many different sizes. The size of the bite block 500 may be determined by the size of the patient. For example, a bite block 500 sized for a newborn may have an endoscopy port diameter 505 of 5 mm. The endoscopy port diameter 505 for an infant, toddler, child, and an adult may all be manufactured to be progressively larger. Endoscopy instruments and tools often fill a majority or an entirety of a cross-sectional area of the endoscopy port 504, and therefore inserting other items, such as an oxygen or a suction tube, through the endoscopy port 504 may not be practical.

The body 502 may include a plurality of secondary ports. The plurality of secondary ports may include a first port 506 and a second port 508. The first port 506 may include a first connector. The first connector may be an oxygen connector. Thus, the first port 506 may be an oxygen port. For example, the first connector may be configured to connect to an oxygen tube. In some embodiments, the first connector may be a standard oxygen connector or a high-flow oxygen connector. For example, the first connector may be the male end of a press-fit oxygen connector, such that an oxygen tube may be inserted over the first connector. In other examples, the first connector may be the female end of a press-fit oxygen connector, such that an oxygen tube may be inserted into an inner chamber of the oxygen connector. In still other examples, an open ended O2 connector with an adapter for a ventilation circuit, such as a connector used in a standard nasal cannula package to attach the cannula to the ventilation circuit may be used. In other embodiments, the first connector may be a mechanically interlocking connector, such as a luer lock connector.

In this manner, oxygen may be delivered to the patient's mouth. During endoscopic procedures, health care providers often deliver oxygen through nasal cannulas. However, in some situations, a patient may not be able to breathe adequately through the nasal passage, or through a nasal cannula, and therefore must breathe through his or her mouth. For example, patients who undergo craniofacial surgery often have limited or no capacity to breathe through their noses, and therefore breathe through their mouths. In other examples, patients who have nasal blockages or other challenges to breathing through their noses may breathe through their mouths. Therefore, oxygen delivered directly to the patient's mouth may enable the patient to receive adequate oxygen during an endoscopic procedure.

Furthermore, some patients may require additional oxygen during an endoscopic procedure. By delivering oxygen directly to the patient's mouth, if a seal between the patient's mouth and the body 502 reduces or eliminates leaks, then the oxygen percentage that the patient breathes may be approximately 90% to 100% oxygen. This may be an increase in oxygen concentration compared to the oxygen concentration delivered through nasal cannula. In some embodiments, a combination of nasal cannula and oxygen delivered directly to the patient's mouth may be used.

In some embodiments, the first connector may be configured to connect to a high-flow oxygen connection. For example, the high-flow oxygen connection may deliver oxygen at a rate of greater than 10 L/min. In other examples, the high-flow oxygen connection may deliver oxygen at a rate of greater than 16 L/min. In still other examples, the high-flow oxygen connection may deliver oxygen at a rate of greater than 20 L/min. In yet other examples, the high-flow oxygen connection may deliver oxygen at a rate of up to 60 L/min. In some embodiments, the high-flow oxygen rate may be controlled to a desired amount and adjusted during an endoscopic procedure. In some embodiments, it may be critical that the high-flow oxygen connection delivers oxygen at a rate of greater than 16 L/min to ensure adequate oxygen flow to the patient during an endoscopic procedure.

The first port 506 may have a first diameter 507. The first diameter 507 may be the same diameter as a standard oxygen tube used in standard or high-flow oxygen. For example, in some embodiments, the first diameter 507 may be between 4 mm and 8 mm. More specifically, the first diameter 507 may be 5 mm or 7 mm. The first diameter 507 may be less than the size of the endoscopy port 504.

The second port 508 may include a second connector. The second connector may be a suction connector. Thus, the second port 508 may be a suction port. For example, the second port 508 and the second connector may be configured to connect to a suction tube. In some embodiments, the second connector may be configured to connect to a soft suction catheter or a soft suction tube. In some embodiments, the second connector may be different from the first connector. For example, the second connector may be a suction connector, and the first connector may be an oxygen connector. In other examples, the second connector may have a larger or a smaller diameter than the first connector. In still other examples, the second connector may have a different shape than the first connector. In some embodiments, the second connector may be a different type of connector than the first connector. For example, the second connector may be an interlocking connector, and the first connector may be a press-fit connector.

The second port 508 may have a second diameter 509. In some embodiments, the second diameter 509 may be larger than the first diameter 507. The second diameter 509 may be any diameter used with suction catheters. For example, the second diameter 509 may be the same as a diameter of the second connector. The second connector may be any gauge used in suction catheters, including 8 Fr, 9 Fr, 10 Fr, 11 Fr, 12 Fr, or any other gauge used in suction catheters. In some embodiments, the second connector may be the male end of a press-fit connection. In other embodiments, the second connector may be the female end of a press-fit connection. In still other embodiments, the second connector may be a mechanical interlock, such as a luer lock. In yet other embodiments, the second connector may be an open port through which a soft suction catheter may be inserted.

The second port 508 may allow a patient to have secretions and other fluids sucked from his or her mouth. This may be performed, for example, with cardiac patients, or other patients who experience an increase in oral secretions. In this manner, the secretions and other fluids may be removed from the mouth and therefore may not interfere with the patient's breathing and/or leak out of the patient's mouth during an operation.

Providing a suction connector or a suction port in the second port 508 may allow a patient to have oxygen delivered simultaneously with suction removal of fluids from the mouth. Furthermore, including a dedicated suction port and oxygen port with a specific oxygen connector and suction connector may enable the suction and the oxygen tubes to remain attached to the body 502. Thus, when the patient is moved, or when a health care provider touches or otherwise moves the oxygen and/or suction tubes, oxygen delivery and suction may remain uninterrupted. Furthermore, dedicated oxygen and suction ports may allow the patient to both have a high-flow of oxygen delivery, and have fluids removed through suction as needed, without requiring a change in tube connections.

The body 502 may further include a third port 510 and a fourth port 512. The third port 510 may have the same diameter as the first port 506. In other words, the third port 510 may have the first diameter 507. The fourth port 512 may have the same diameter as the second port 508. In other words, the fourth port 512 may have the second diameter 509.

In some embodiments, the first port 506 and the second port 508 may be located on a first side 514 of the endoscopy port 504, and the third port 510 and the fourth port 512 may be located on a second side 516 of the endoscopy port 504. The first side 514 may be located opposite the endoscopy port 504 from the second side 516. For example, the first side 514 may be located on a right side of the endoscopy port 504, and the second side 516 may be located on a left side of the endoscopy port 504. In other examples, the second side 516 may be mirrored across a center of the endoscopy port from the first side 514. In some embodiments, the first port 506 and the second port 508 may be offset in a first direction from the endoscopy port 504. The first port 506 may be offset with the same amount as the second port 508, or the first port 506 may be offset with a different amount than the second port 508. In the same or other embodiments, the third port 510 and the fourth port 512 may be offset in a second direction from the endoscopy port 504. The second direction may be diametrically opposite, or 180° different from, the first direction. The third port 510 and the fourth port 512 may be offset with the same or different amounts.

In some embodiments, the first port 506 and the third port 510 may be located at a first height from an upper surface 511 of the body 502. In other words, the first port 506 and the third port 510 may be located the same distance from the upper surface 511 of the body 502. In other embodiments, the first port 506 and the third port 510 may be located at different heights or distances from the upper surface 511. In some embodiments, the second port 508 and the fourth port 512 may be located at a second height from the upper surface 511 of the body 502. In other words, the second port 508 and the fourth port 512 may be located the same distance from the upper surface 511 of the body 502. In other embodiments, the second port 508 and the fourth port 512 may be located at different distances or heights from the upper surface 511.

In some embodiments, the third port 510 may include the first connector. In other words, the third port 510 may include an oxygen connector and may be an oxygen port. The fourth port 512 may include the second connector. In other words, the fourth port 512 may include a suction connector and may be a suction port. In this manner, the first port 506 and the third port 510 may both be oxygen ports, and the second port 508 and the fourth port 512 may both be suction ports. This may allow for redundancy between the ports. Depending on the patient's anatomy, body position, and other factor, the second port 508 or the fourth port 512 may provide better access to the collected fluids for suction.

Any combination of ports may be used simultaneously during a procedure. For example, the first port 506 and the second port 508 may be used, while the third port 510 and the fourth port 512 are inactive. In some embodiments, inactive ports may be closed with a stopper. In other examples, the third port 510 and the fourth port 512 may be used, while the first port 506 and the second port 508 are inactive. In yet other examples, the first port 506 and the fourth port 512 may be used, while the second port 508 and the third port 510 are inactive. In still other examples, the second port 508 and the third port 510 may be used, while the first port 506 and the fourth port 512 are inactive. In further examples, any combination of three ports may be used, with a single port being inactive. In still further examples, all four ports may be used, with no port being inactive. In some embodiments, the port being used may be switched in the middle of a procedure.

The body 502 may include a plurality of flanges 518. The flanges 518 may be configured to connect to a strap that may be wrapped around a patient's head to secure the bite block 500 to the patient's head. In the embodiment shown in FIG. 5A, the flanges 518 may be a hole in the body 502. In other embodiments, the flanges 518 may be protrusions or extensions extending out of the sides of the body 502.

FIG. 5B is a top-down representation of the bite block 500 of FIG. 5A. The body 502 may include an outer surface 520 (e.g., the surface shown in FIG. 5A) and an inner surface 522. A plurality of ports (e.g., endoscopy port 504, first port 506, second port 508, third port 510, and fourth port 512 of FIG. 5A) may extend through the body 502 from the outer surface 520 to the inner surface 522.

The plurality of ports may connect to a plurality of tubes that extend past the inner surface 522. For example, an endoscopy port may connect to an endoscopy tube 524 that extends past the inner surface 522. The endoscopy tube 524 may be sized to allow an endoscopy instrument or tool to be inserted into the patient's mouth, and to protect the mouth and/or the instrument from damage.

The first port may connect to a first tube 526 that extends past the inner surface 522. The second port may connect to a second tube 528 that extends past the inner surface 522. The third port may connect to a third tube 530 that extends past the inner surface 522. The fourth port may connect to a fourth tube 532 that extends past the inner surface 522.

The first tube 526 and the second tube 528 may extend past the inner surface 522 for a first distance 534. The third tube 530 and the fourth tube 532 may extend past the inner surface 522 for a second distance 536. In some embodiments, the first distance 534 may be different than the second distance 536. For example, the first distance 534 may be less than the second distance 536. In this manner, oxygen may be delivered through the first tube 526 and/or the third tube 530 to different locations in the patient's mouth. Similarly, suction may occur through the second tube 528 and/or the fourth tube 532 at different locations in the patient's mouth. Different patients may have different anatomies, different postures during a procedure, different sized mouths, or otherwise have different structures that may require different suction and/or oxygen locations. For example, a patient may collect fluid at a back of the mouth, and the fourth tube 532 may be better situated to suction fluid from the back of the mouth. In another example, a patient may collect fluid at a front of the mouth and the second tube 528 may be better situated to suction fluid from the front of the mouth.

In some embodiments, the endoscopy tube 524 may extend past the inner surface 522 for the first distance 534. In other words, the endoscopy tube 524 may have the same length as the first tube 526 and the second tube 528. In other embodiments, the endoscopy tube 524 may extend past the inner surface 522 for the second distance 536. In still other embodiments, the endoscopy tube 524 may be any length, including longer than the second distance 536, shorter than the first distance 534, or between the first distance 534 and the second distance 536.

The bite block 500 may include a tongue protector 538. The tongue protector 538 may extend past the inner surface 522. In some embodiments, the tongue protector 538 may extend past the inner surface 522 for the second distance 536. In other embodiments, the tongue protector 538 may extend past the inner surface 522 further than the second distance 536. The tongue protector 538 may protect the tongue from endoscopy instruments and tools and/or may keep the tongue from blocking the patient's airway.

In at least one embodiment, the second tube 528 and the fourth tube 532 may be connected to the tongue protector 538. This may anchor the second tube 528 and the fourth tube 532 to the tongue protector 538, which may keep the second tube 528 and the fourth tube 532 from moving around inside the patient's mouth. The first tube 526 may be connected to the second tube 528 and the third tube 530 may be connected to the fourth tube 532. In the same or other embodiments, the endoscopy tube 524 may be connected to the tongue protector 538. In this manner, the plurality of tubes of the bite block 500 may be anchored to the tongue protector 538, and be prevented from moving around in the mouth.

In some embodiments, the endoscopy tube 524 may be fabricated from a hard material, such as an extruded plastic, an injection molded plastic, a polymeric material, hard rubber, a composite material, or a metal or metallic alloy. In this manner, the endoscopy tube 524 may be rigid, and allow the patient's teeth to engage the endoscopy tube 524 during an endoscopic procedure.

In some embodiments, the endoscopy tube 524 may have an endoscopy opening 540, the first tube 526 may have a first opening 542, the second tube 528 may have a second opening 544, the third tube 530 may have a third opening 546, and the fourth tube 532 may have a fourth opening 548. The endoscopy opening 540, the first opening 542, the second opening 544, the third opening 546, and the fourth opening 548 may all be different. In other words, the endoscopy opening 540, the first opening 542, the second opening 544, the third opening 546, and the fourth opening 548 may all open to a different location. For example, open space 550 may exist past the inner surface 522 of the body 502. The endoscopy opening 540, the first opening 542, the second opening 544, the third opening 546, and the fourth opening 548 may all open into the open space 550. In other words, none of the first opening 542, the second opening 544, the third opening 546, and the fourth opening 548 may open into the endoscopy tube 524, or into any other of the plurality of tubes. In this manner, each of the endoscopy opening 540, the first opening 542, the second opening 544, the third opening 546, and the fourth opening 548 may be able to access a different part of the patient's mouth.

FIG. 5C is a side view of the bite block 500 of FIG. 5A, according to at least one embodiment of the present disclosure. In some embodiments, the body 502 may be curved to match the profile of a patient's mouth. In other embodiments, the body 502 may be straight. As may be seen in FIG. 5C, the first tube 526 and the second tube 528 may be the same length as the endoscopy tube 524. The third tube and the fourth tube 532 may be longer than the first tube 526, the second tube 528, and the endoscopy tube 524. The second tube 528, the fourth tube 532, and the endoscopy tube 524 may be connected to the tongue protector 538. The first tube 526 may be connected to the second tube 528 and the third tube 530 may be connected to the fourth tube 532. In this manner, all of the tubes may be fixed to the tongue protector 538.

FIG. 5D is another side view of the bite block 500 of FIG. 5A. As may be seen, the third tube 530 and the fourth tube 532 may be longer than the endoscopy tube 524.

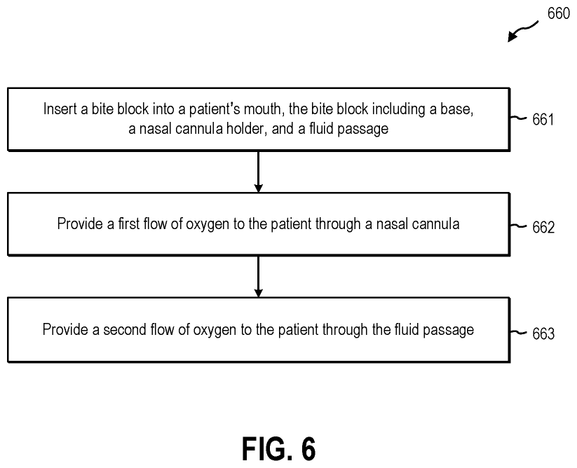

FIG. 6 illustrates a flowchart of a method 660 or a series of acts for using a bite block according to one embodiment of the present disclosure. While FIG. 6 illustrates acts according to one embodiment, alternative embodiments may omit, add to, reorder, and/or modify any of the acts shown in FIG. 6. The acts of FIG. 6 can be performed as part of a method.

The method 660 may include inserting a bite block into a patient's mouth at 661. The bite block may include a base, a probe opening, a nasal cannula holder, and a fluid passage. The nasal cannula holder may be connected to an upper portion of the base and may be configured to retain a nasal cannula. In one embodiment, the fluid passage may be a first fluid passage and the bite block may include a second fluid passage. In another embodiment, the method 660 may include inserting an oral airway body into the patient's mouth through the probe opening at 661. The oral airway body may be inserted into the patient's mouth while the base is already inserted and/or in place in the patient's mouth. The oral airway may include the first and second fluid passages. The method 660 may also include detachably connecting the oral airway body to the base at 661. In this way, the base may be used in a medical procedure with or without the oral airway body.

The method 660 may include providing a first flow of oxygen to the patient through a nasal cannula at 662. The nasal cannula may be connected to the nasal cannula holder. In this way, a nasal cannula may be used in connection with use of the bite block to provide oxygen to a patient. The method 660 may also include providing a second flow of oxygen to the patient through the fluid passage at 663. The second flow of oxygen may be distinct from the first flow of oxygen and may follow a distinct fluid path from that of the first flow of oxygen. In some situations, different portions of a medical procedure may include different oxygenation requirements and/or different oxygen uptake mechanisms. By providing two distinct flows of oxygen to the patient through the fluid passage and the nasal cannula, a healthcare provider may tailor the oxygen flow to the patient based on the operation being performed.

Additionally, the method 660 may include providing the second flow of oxygen directly to the airway of the patient at 663. Oxygen provided directly to a patient's airway may be more effective at maintaining an adequate blood oxygenation level that nasal oxygen. In this way, providing oxygen through the fluid passage directly to the airway of the patient may provide a more effective oxygen flow than the first flow of oxygen.

As discussed above, the fluid passage may be a first fluid passage and the bite block may include a second fluid passage. The method 660 may also include providing a suction flow to the patient through the second fluid passage at 663. The second fluid passage may similarly provide suction directly to the airway of a patient. In this way, secretions may be removed directly from the patient's airway, which may prevent complications that may trigger a laryngospasm, asphyxiation, or other medical condition during an oral procedure.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A bite block for use during an endoscopic operation, comprising:
a base;
a probe opening through the base;
an upper biting platform connected to an upper portion of the base;
a lower biting platform connected to a lower portion of the base;
a nasal cannula holder configured to retain a nasal cannula, and connected to the upper portion of the base;
a first fluid passage, the first fluid passage having a first proximal end at the base and a first distal end extending from the base such that, when the bite block is in use with a patient, the first distal end is configured to extend to a throat of said patient, the first fluid passage defining a first fluid path extending between the first proximal end and the first distal end; and
a second fluid passage, the second fluid passage having a second proximal end at the base and a second distal end extending from the base such that, when the bite block is in use with said patient, the second distal end is configured to extend to the throat of said patient, the second fluid passage defining a second fluid path extending between the second proximal end and the second distal end.

2. The bite block of claim 1, wherein the first fluid passage includes a first connector and the first connector is an oxygen adapter, and wherein the first fluid path is an oxygen fluid path.

3. The bite block of claim 1, wherein the first fluid passage includes a first connector that is angled away from the probe opening.

4. The bite block of claim 1, wherein the second fluid passage includes a second connector, the second connector is a suction adapter, and the second fluid path is a suction fluid path.

5. The bite block of claim 1, wherein:
the first fluid passage includes a first connector, the first connector being an oxygen adapter;
the second fluid passage includes a second connector, the second connector being an oxygen adapter; and
the first fluid path is a first oxygen fluid path and the second fluid path is a second oxygen fluid paths.

6. The bite block of claim 1, wherein the first fluid path is a high-flow oxygen fluid path.

7. The bite block of claim 1 wherein the nasal cannula holder is configured to retain the nasal cannula in a position that is inserted in nostrils of said patient to deliver a flow of nasal oxygen.

8. The bite block of claim 1, wherein the nasal cannula holder is configured to retain the nasal cannula without impeding a flow of oxygen through the nasal cannula.

9. The bite block of claim 1, wherein the first fluid passage is configured to provide an oral oxygen flow to said patient along a distinct fluid path from the nasal cannula.

10. A method for treating a patient, comprising:
inserting a bite block into a mouth of said patient, the bite block including a base, a probe opening, a nasal cannula holder configured to retain a nasal cannula, the nasal cannula holder connected to an upper portion of the base, and a fluid passage;
providing a first flow of oxygen to said patient through the nasal cannula connected to the nasal cannula holder; and
providing a second flow of oxygen to said patient through the fluid passage, wherein the second flow of oxygen is distinct from the first flow of oxygen.

11. The method of claim 10, wherein the fluid passage is a first fluid passage and further comprising providing a suction flow to said patient through a second fluid passage.

12. The method of claim 10, wherein providing the second flow of oxygen further includes providing the second flow of oxygen directly to an airway of said patient.

13. The method of claim 10, further comprising:
inserting an oral airway body through the probe opening and into said patient's mouth, the oral airway body including the fluid passage and a second fluid passage; and
detachably connecting the oral airway body to the base.

14. A bite block for use during an endoscopic operation, comprising:
a base, including:
a probe opening through the base;
an upper biting platform connected to an upper portion of the base;
a lower biting platform connected to a lower portion of the base;
a nasal cannula holder configured to retain a nasal cannula, and connected to the upper portion of the base; and
an oral airway, including:
a first fluid passage;
a second fluid passage;
an anterior portion; and
a posterior portion configured to rest on a base of a tongue of a patient using the bite block.

15. The bite block of claim 14, wherein the oral airway is curved downward and is configured to pull the tongue of the patient forward to provide posterior oral access.

16. The bite block of claim 14, wherein the posterior portion further comprises a first portion and a second portion with a gap therebetween.

17. The bite block of claim 16, wherein the first fluid passage passes through the first portion and the second fluid passage passes through the second portion.

18. The bite block of claim 16, wherein the gap is configured to guide a medical device during oral application of the medical device.

19. The bite block of claim 14, wherein the oral airway is detachably connected to the base.

20. The bite block of claim 14, wherein an oral airway width is less than a probe opening width such that the oral airway may fit through the probe opening.
